(19) United States
(12) Patent Application Publication
(10) Pub. No.: US 2026/0283946 A1
(43) Pub. Date: Sep. 24, 2026

(54) PHARMACEUTICAL DRY POWDER COMPOSITION AND METHODS FOR TREATING RESPIRATORY TRACT INFECTIONS

(71) Applicant: Liita Care ApS, København K (DK)

(72) Inventors: Suzana Erico Tanni MINAMOTO, København K (DK); Martin OHRT, København K (DK)

(21) Appl. No.: 18/997,966

(22) PCT Filed: Sep. 2, 2022
(86) PCT No.: PCT/EP2022/074500

§ 371 Date: Jan. 23, 2025

(51) Int. Cl. A61K 9/00; A61K 33/14; A61K 47/26; A61P 31/14

(52) U.S. Cl. CPC A61K 9/0075; A61K 33/14; A61K 47/26; A61P 31/14

(57) ABSTRACT

The present invention provides a pharmaceutical dry powder composition for use in treating, preventing and/or amelio-rating viral infection in airways of a mammal, such as a human subject, said composition comprising micronized sodium salt as the main component and primary therapeutic agent. The present invention further provides a pharmaceutical composition for use in treating and/or ameliorating cough. The subject to be treated with the pharmaceutical composition may be infected with SARS-COV-2 and/or diagnosed with COVID-19 infection. The pharmaceutical composition is administered by dry powder inhalation.

### FIELD OF THE INVENTION

The present invention concerns a pharmaceutical composition for use in treating, preventing and/or ameliorating viral infection in airways of a mammal, such as a human subject. The present invention further concerns a pharmaceutical composition for use in treating and/or ameliorating cough of a mammal, such as a human subject, such as cough caused by a viral infection in the airways of the subject. The human subject to be treated with the pharmaceutical composition may be a subject infected with SARS-COV-2 and/or diagnosed with COVID-19 infection. The pharmaceutical composition of the present invention is a dry powder composition comprising micronized sodium salt as the main component and primary therapeutic agent. The pharmaceutical composition is administered by dry powder inhalation.

### BACKGROUND OF THE INVENTION

Respiratory tract infections are common infections of the upper respiratory tract (e.g., nose, ears, sinuses, and throat) and the lower respiratory tract (e.g., trachea, bronchial tubes, and lungs). Symptoms of upper respiratory tract infections include runny or stuffy nose, irritability, restlessness, poor appetite, decreased activity level, coughing, and fever. Viral infections of the upper respiratory tract cause, or are associated with, for example, sore throats, colds, croup, and the flu. Clinical manifestations of a lower respiratory tract infection include shallow coughing that produces sputum in the lungs, fever, and difficulty breathing.

Respiratory viral infections cause an enormous disease burden in infants, children and adults. In persons with underlying cardiopulmonary disease conditions the clinical impact of common infections is even greater. Current therapies for viral respiratory tract infections involve the administration of anti-viral agents for the treatment, prevention, or amelioration.

Some common classes of human disease associated respiratory viruses include: paramyxoviruses, orthomyxoviruses, adenoviruses, picornavirus, parvoviruses, arenaviruses, herpesviruses, retroviruses, and coronaviruses.

Severe acute respiratory syndrome-related coronavirus (SARSr-COV or SARS-COV) is a species of virus consisting of many known strains. The SARSr-COV species is a member of the genus Betacoronavirus and of the subgenus Sarbecovirus (SARS Betacoronavirus). The morphology of the SARS-related coronavirus is characteristic of the coronavirus family as a whole. These viruses are enveloped, positive-sense single-stranded RNA viruses. SARS-related coronavirus follows the replication strategy typical of all coronaviruses. Two strains of the virus have caused outbreaks of severe respiratory diseases in humans: severe acute respiratory syndrome coronavirus 1 (SARS-COV or SARS-COV-1), which caused the 2002-2004 outbreak of severe acute respiratory syndrome (SARS), and severe acute respiratory syndrome coronavirus 2 (SARS-COV-2), which is causing the ongoing pandemic of COVID-19.

Viral infections are frequently highly contagious, especially when spread by respiration. The recent pandemics caused by severe acute respiratory syndrome-related coronavirus are proof of how rapidly an infection can spread worldwide.

Potential ways in which the virus can be spread include touching the skin of other people or objects that are contaminated with infectious droplets and then touching your eye(s), nose, or mouth. Further, airborne infection is also a likely route of viral transmission. It is also possible that the virus can be spread more broadly through the air via aerosols composed of mucus droplets originating from the lungs and/or nasal cavities produced when a human or animal coughs or simply breathes. These aerosols can contain virus whereby the disease is transmitted upon inhalation by exposed subjects.

Corona virus may cause both upper and lower respiratory tract infections.

Although several means of treating and/or preventing viral infections are available, there is a great demand for new treatments to address the increasing health care issues such as new pathogen strains arising, and high cost of traditional pharmaceuticals.

The present invention encompasses methods to reduce virus growth, infectivity, burden, shed, and development of antiviral resistance, and to enhance the efficacy of traditional anti-viral therapies.

### SUMMARY OF THE INVENTION

In one aspect, the present invention provides a dry powder composition comprising at least 50 wt %, such as at least 60 wt %, such as at least 70 wt %, such as at least 80 wt %, such as at least 90 wt % NaCl for use in preventing and/or treating a viral respiratory tract infection, such as coronavirus, such as SARS-COV-2, wherein said composition is administered to a mammalian subject by dry powder inhalation, such as by using a dry powder inhaler.

In one embodiment, the dry powder composition of the present invention reduces the time for resolution of one or more symptoms, such as cough, caused by the viral respiratory tract infection.

In one embodiment, the dry powder composition of the present invention has a particle size distribution of at least 80% of the particles being in the range 1-10 μm. In one embodiment, the dry powder composition of the present invention further comprises an anti-agglomerating agent, such as a carbohydrate or carbohydrate derivative, such as lactose, mannitol, and maltodextrin, preferably lactose.

In one embodiment, the dry powder composition of the present invention substantially consists of NaCl and lactose. In one embodiment, the NaCl to lactose ratio is between 99:1 and 75:25 based on wt %.

In one embodiment, the sole therapeutic agent(s) of the dry powder composition of the present invention is NaCl, or NaCl in combination with the anti-agglomerating agent.

In one embodiment, the dry powder composition of the present invention is administered in a daily dosage of between 0.5-200 mg. In one embodiment, the daily dosage is divided into 1-10 daily sessions of 4-10 mg per session.

In one embodiment, the dry powder composition of the present invention is administered orally and/or nasally. In one embodiment, the dry powder composition of the present invention is administered to a human subject.

In one embodiment, the dry powder composition of the present invention reduces the time for resolution of cough of the mammalian subject by at least 30% compared to a mammalian subject not being treated with the dry powder composition.

In one embodiment, the NaCl of dry powder composition of the present invention is provided by a composition obtained from the Hvornum Salt Diapir (N56 36.834 E009 42.070) in Denmark.

### DESCRIPTION OF THE INVENTION

### Brief Description of the Figures

FIG. 1: Average reduction (%) of SARS-COV-2 viral RNA (y-axis) in the supernatant of CCL-81 VERO cells under treatment with different concentrations of BREATHOX®POWDER (x-axis). Vero cells were pretreated with different concentrations of BREATHOX® POWDER for 1 hour prior to virus infection, followed by incubation with virus for 1 hour in the presence of the BREATHOX® POWDER.

FIG. 2: Luciferase assay, measuring intracellular ATP concentration via luminescence production, in cells treated with different concentrations of BREATHOX® POWDER after 1 h, 24 h and 72 h.

FIG. 3: Indirect measure of total cellular ATP concentration. The total ATP concentration of Vero cells treated for 1 h with increasing BREATHOX® POWDER concentrations was measured by the luciferase assay. BREATHOX® POWDER was diluted in cell culture medium containing 0.6% NaCl (110 mM NaCl), with our without ouabain (5 μM). Data are plotted as average with standard error bars of 12 replicates (*p<0.05). Sham=cells in the presence of increasing concentrations of BREATHOX® POWDER (white bars); Ouabain=cells in the presence of ouabain and increasing concentrations of BREATHOX® POWDER (grey bars).

FIG. 4: Mitochondrial status of cultured Vero cells (40 000 cells/well) treated with different concentrations of BREATHOX® POWDER (0, 0.2, 0.4, 0.8, 0.9, 1.1, 1.2, 1.4% wt/vol). (A) The oxygen consumption rate, OCR and (B) Extracellular acidification rate, ECAR were measured using Seahorse technology with the Mito Stress Test kit. Sequential injections of oligomycin, carbonyl cyanide 3-chlorophenylhydrazone (CCCP), and rotenone plus antimycin A of the cultured cells are indicated. The ECAR rate was verified by application of the glycolysis stress test kit (Agilent Technologies). The data are representative of three independent experiments and shown as mean values+SEM; two-way ANOVA (*p≤0.05). (C) The correlation between OCR and ECAR is plotted as an energy map. (D) General illustration of mitochondrial respiration evaluation under four different conditions: (i) basal respiration (corresponding to the cell basal consumed oxygen); (ii) proton leak (after oligomycin addition (ATP-synthase blocker)); (iii) uncoupled (after addition of the respiratory chain uncoupler FCCP, where the oxygen consumed reflects the maximal respiration rate, irreversibly uncoupling from ATP synthesis); and (iv) inhibited, through complex I and complex III total inhibition by rotenone and antimycin A, respectively.

FIG. 5: Intracellular Na+ concentration variation (Δ[Na+]i) in Vero cells. Vero cells cultured in a medium comprising 0.6% NaCl were incubated with Natrium Green (11 μM) and Pluronic Acid (0.07%) for 45 min. BREATHOX® POWDER was added to the cell cultures in different concentrations. Changes in Na+ concentration were recorded by inverted fluorescence microscope Nikon Eclipse Ti coupled to an Andor CCD camera. (A) Response lines of cells representing the cellular sodium inward flow detected by the natrium green dye over time, for three different BREATHOX® POWDER concentrations (0.4, 0.8, 1.1% wt/vol). The arrow indicates the time point of BREATHOX® POWDER application. (B) Data from (A) plotted as averages with standard error bars of 70 cells/10 2 replicates.

FIG. 6: Real time PCR assay of Ato1B1 mRNA encoding Na+/K+ ATPase channel protein in Vero cells after 72 hours of BREATHOX® POWDER incubation.

FIG. 7: Study design for testing BREATHOX® antiviral effects in COVID-19 patients.

FIG. 8: Cough as a symptom of Covid-19 affecting the airways. Recovery time from the beginning of the symptoms of patients treated with BREATHOX® 5 sessions/day for 10 days (group 1), BREATHOX® 10 sessions/day for 10 days (group 2), or standard treatment of care (group 3). 1 session=4 inhalations. Y-axis shows fraction of patients experiencing cough (1.00=100%), at the time indicated on the x-axis. Value below graph indicate number of patients in each group experiencing cough at the time indicated on the x-axis on the graph. (A) Data plot of first 10 days of treatment. (B) Same data as in (A), but extended to show 20 days.

FIG. 9: Inhaler suitable for administering the dry powder composition of the present invention. (A) a cross-sectional top view of the inhaler in ‘CLOSED’ position and in ‘OPEN’ position, (B) a perspective side view of the inhaler in the ‘CLOSED’ position and in the ‘OPEN’ position, (C) an exploded view of the inhaler.

### ABBREVIATIONS, TERMS AND DEFINITIONS

The term “respiratory tract” as used herein includes the upper respiratory tract (e.g., nasal passages, nasal cavity, throat, pharynx), respiratory airways (e.g., larynx, tranchea, bronchi, bronchioles) and lungs (e.g., respiratory bronchioles, alveolar ducts, alveolar sacs, alveoli).

The term “respiratory tract infection” is a term of art that refers to upper respiratory tract infections (e.g., infections of the nasal cavity, pharynx, larynx) and lower respiratory tract infections (e.g., infections of the trachea, primary bronchi, lungs) and combinations thereof. Typical symptoms associated with respiratory tract infections include nasal congestion, cough, running nose, sore throat, fever, facial pressure, sneezing, chest pain and difficulty breathing.

The term “dry powder” as used herein refers to a composition containing finely dispersed respirable dry particles that are capable of being dispersed in an inhalation device and subsequently inhaled by a subject. Such dry powder(s) or dry particle(s) is substantially free of water or other solvent, or is anhydrous (i.e. no water present), to avoid agglomeration. The dry powder of the present invention may be referred to as “NaCl powder”, which should be understood as a dry powder composition comprising NaCl according to the disclosures herein.

The term “micronized” a used herein in relation to a dry powder composition refers to a dry powder having particles of a size suitable for inhalation through the airways of a mammal, such as a human. Typically, such particle size is between 0.1 μm and 20 μm. The term “micronized” is not limited to the micronization process as such.

The term “aerosol” as used herein refers to any preparation of a fine cloud of particles. For the present invention, specifically non-liquid particles—i.e. dry powders make up the aerosol. Typically the fine cloud of particles in the aerosol have a volume median geometric diameter of about 0.1 to about 30 microns or a mass median aerodynamic diameter of between about 0.5 and about 10 microns.

The term “excipient” as used herein means any suitable compound for supporting and/or increasing/improving the dryness and/or flowability of the sodium chloride powder.

The term “anti-agglomeration agent” as used herein means a compound that prevents agglomeration of the dry powder composition and in particular the sodium chloride, such as sugars, in particular micronized sugars, in particular lactose.

The term “viral infection” as used herein means any clinical manifestation or symptom caused by the entry of viruses that are inhaled into the airways of a mammal, such as a human. The viral particles may be deposited in the nasal or oral cavity, pharynx, trachea, primary bronchi, secondary bronchi, terminal bronchi, and/or the alveoli.

The term “standard care of treatment” as used herein refers to paracetamol.

The term “treatment” and “treating” as used herein means the management and care of a patient for the purpose of combating a condition, such as a disease or a disorder. The term is intended to include the full spectrum of treatments for a given condition from which the patient is suffering, such as administration of the dry powder composition of the present invention to alleviate the symptoms or complications, to delay the progression of the disease, disorder or condition, to alleviate or relief the symptoms and complications, and/or to cure or eliminate the disease, disorder or condition as well as to prevent the condition, wherein “prevention” is to be understood as the management and care of a patient for the purpose of combating the disease, condition, or disorder and includes the administration of the dry powder composition of the present invention to prevent the onset of the symptoms or complications.

The terms “a” and “an” and “the” and similar referents as used in the context of describing the invention are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein. Unless otherwise stated, all exact values provided herein are representative of corresponding approximate values (e.g., all exact exemplary values provided with respect to a particular factor or measurement can be considered to also pro-vide a corresponding approximate measurement, modified by “about,” where appropriate).

### DETAILED DESCRIPTION OF THE INVENTION

The present invention concerns a pharmaceutical composition for use in treating, preventing and/or ameliorating viral infection in airways of a mammal, such as a human subject. In preferred embodiments, the human subject to be treated with the pharmaceutical composition is a subject infected with SARS-COV-2 and/or diagnosed with COVID-19 infection.

The present invention more specifically relates to an inhalable dry composition comprising sodium chloride (NaCl) for use in therapy. Use of the composition facilitates the prevention or reduction of viral growth, and the inhibition of viral replication in the airways of a mammal, such as human.

The present invention further relates to an inhalable dry composition comprising sodium chloride (NaCl) for use in reducing of duration of symptoms in viral infections. Typical symptoms are selected form one or more of fever, shortness of breath, nasal blockage, cough, sore throat, runny nose, sneezing, fever, fatigue, body ache, and chest pain. In a further embodiment, the dry composition comprising sodium chloride facilitates reduction of post-covid symptoms.

The composition may be inhalable through oral and/or nasal route-hence the dry powder composition comprises inhalable particles of sodium chloride of a size suitable for getting into the airways of a mammal. In preferred embodiments, the dry powder of sodium chloride is a micronized dry powder.

Inhalation therapy is capable of providing a drug delivery system that is easy and safe to use in an inpatient or outpatient setting. The present invention can preferably replace treatments using nebulized formulations, which thereby ultimately protects healthcare workers and other subjects in close proximity to the patient, as nebulization can potentially expose others to contaminated aerosols.

Various embodiments of the invention are described below. The embodiments should be seen as referring to any one of the aspects described herein as well as any one of the embodiments described herein, unless it is specified that an embodiment relates to a specific aspect or aspects of the present invention.

All headings and sub-headings are used herein for convenience only and should not be construed as limiting the invention in any way.

### I. Pharmaceutical Dry Powder Formulation

In one aspect, the present invention provides a pharmaceutical inhalable dry powder formulation comprising NaCl as its main component.

### I.i Composition of the Pharmaceutical Dry Powder Formulation

NaCl is the main component of the pharmaceutical dry powder formulation.

Without wishing to be bound by theory, the present inventors have found that sodium chloride decreases intracellular ATP concentration; this being one mechanism to explain how the dry powder composition of the present invention decreases replication of SARS-CoV-2 in mammalian cells. Treating Vero cells with BREATHOX® POWDER in presence or absence of Ouabain (Na+/K+ ATPase transporter inhibit), it was found that the reduced level of ATP was caused by the increased activity of the cells Na+/K+ ATPase transporter (see Example 2).

In one embodiment, the amount of sodium chloride in the dry powder composition is at least 50, 51, 52, 53, 54, 55, 56, 57, 58, 59, 60, 61, 62, 63, 64, 65, 66, 67, 68, or 69 weight %, preferably at least 70, 71, 72, 73, 74, 75, 76, 77, 78, or 79 weight %, more preferably at least 80, 81, 82, 83, 84, 85, 86, 87, 88, or 89 weight %, most preferably at least 90, 91, 92, 93, 94, 95, 96, 97, 98, or 99 weight %.

In one embodiment, the amount of sodium chloride in the dry powder composition is between 70-99 weight %, between 71-99 weight %, between 72-99 weight %, between 73-99 weight %, between 74-99 weight %, between 75-99 weight %, between 76-99 weight %, between 77-99 weight %, between 78-99 weight %, between 79-99 weight %, between 80-99 weight %, between 81-99 weight %, between 82-99 weight %, between 83-99 weight %, between 84-99 weight %, between 85-99 weight %, between 86-99 weight %, between 87-99 weight %, between 88-99 weight %, between 89-99 weight %, between 90-99 weight %, between 91-99 weight %, between 92-99 weight %, between 93-99 weight %, between 94-99 weight %, between 95-99 weight %, between 96-99 weight %, between 97-99 weight %, or between 98-99 weight %. In one embodiment, the amount of sodium chloride in the dry powder composition is between 89-99 weight %, between 90-98 weight %, between 91-97 weight %, between 92-97 weight %, between 93-97 weight %, between 94-96 weight %, such as preferably around 95 weight %.

A high weight % of NaCl in the dry powder composition ensures delivery of a high concentration of NaCl locally at a point of interest, such as within the respiratory tract, as disclosed in greater detail herein. A targeted delivery of high concentrations of NaCl is advantageous because the higher the concentration of NaCl, the greater the osmotic power of the compound. In Example 1, it was also shown that as the concentration of BREATHOX® POWDER increased, SARS-COV-2 replication was reduced in proportion to the concentration of BREATHOX®. The higher the concentration of BREATHOX® POWDER, the greater the reduction in viral replication.

In one embodiment, the sodium chloride has pharmaceutical quality, such as SANAL® P+ (available from Dansk Salt A/S). Typically, the sodium chloride is SANAL® (Permian raw salt manufactured according to GMP-ICH Q7). Preferably, the sodium chloride is extracted from the Hvornum Salt Diapir (N56 36.834 E009 42.070), Denmark; such as the sodium chloride is extracted from the Danish underground by Maricogen A/S and/or by Nouryon. Salt compositions from the Hvornum Salt Diapir are known for their very high purity—this is of relevance as salt in its purest form and without additives plays an essential role in the pharmaceutical industry. In one preferred embodiment, the NaCl of the dry powder composition of the present invention is provided by a composition obtained from the Hvornum Salt Diapir (N56 36.834 E009 42.070) in Denmark.

In one embodiment the sodium chloride can be manufactured in compliance with the Monograph Sodium Chloride's current version no. 193 of the European Pharmacopoeia. Micronized sodium chloride is the main component of the pharmaceutical dry powder formulation. The inhalable dry powder composition may contain further powders, preferably micronized powders, of other salts and/or minerals and/or excipients in minor amounts.

Excipient carrier particles can be part of the pharmaceutical formulation and be co-delivered with the therapeutic aerosol to aid in achieving efficient aerosolization among other possible benefits. In one embodiment, the dry powder composition comprises an excipient. In a particularly preferred embodiment, the excipient is an anti-agglomeration agent. In one embodiment, the excipient is an anti-agglomeration agent is a carbohydrate. In one embodiment, the excipient is an anti-agglomeration agent is a sugar or sugar derivative. In one embodiment, the excipient is an anti-agglomeration agent selected from a disaccharide, a sugar alcohol, and a polysaccharide. In one embodiment, the anti-agglomeration agent selected from different amino acids. In one preferred embodiment the anti-agglomeration agent is selected from lactose, mannitol and maltodextrin.

An anti-agglomerization agent, such as lactose, amino acids (e.g. leucin) and manitol, are added to enhance the dry powder's flowability, reduce the salt particles' agglomeration due to external factors, and partially act as a carrier.

In one embodiment, the amount of anti-agglomeration agent, such as lactose, in the dry powder composition is at most 50, 49, 48, 47, 46, 45, 44, 43, 42, 41, 40, 39, 38, 37, 36, 35, 34, 33, 32, or 31 weight %, preferably at most 30, 29, 28, 27, 26, 25, 24, 23, 22, or 21 weight %, more preferably at most 20, 19, 18, 17, 16, 15, 14, 13, 12, or 11 weight %, even more preferably at most 10, 9, 8, 7, 6, 5, 4, 3, 2 or 1 weight %. In one embodiment, the amount of anti-agglomeration agent, such as lactose, in the dry powder composition is between 1-10%, 2-8%, 3-7%, 4-6% or preferably around 5 weight %.

In one preferred embodiment, the anti-agglomeration agent is lactose. In one embodiment, the lactose is Alpha-lactose monohydrate of pharmaceutical grade and conforms with European Pharmacopoeia, such as CAS number 7647-14-5.

In one embodiment, the dry powder composition essentially consists of NaCl and lactose. In one embodiment, NaCl and lactose together make up at least 80, 81, 82, 83, 84, 85, 86, 87, 88, or at least 89 weight % of the total dry powder composition, preferably at least 90, 91, 92, 93, 94, or at least 95 weight % of the total dry powder composition, more preferably at least 95, 96, 97, 98 or at least 99% of the total dry powder composition; such as at least 99.1, 99.2, 99.3, 99.4, 99.5, 99.6, 99.7, 99.8, 99.9 or even 100 weight % of the total dry powder composition. In one embodiment, the dry power composition of the present invention consists of NaCl and lactose.

In one embodiment, the weight ratio between the sodium chloride and the anti-agglomeration agent, such as lactose, is from 99/1 to 50/50, 99/1 to 55/45, 99/1 to 60/40, 99/1 to 65/35, or 99/1 to 70/30, 99/1 to 75/25, 99/1 to 70/30, 99/1 to 85/25, 99/1 to 90/10, 98/2 to 90/10, 97/3 to 90/10, 96/4 to 90/10, 95/5 to 90/10, 99/1 to 95/5, 98/2 to 95/5, 97/3 to 95/5, or from 96/4 to 95/5. Preferably, the weight ratio between the sodium chloride and lactose is approximately 95/5.

In one embodiment, the dry powder composition comprises between 50-99 weight % NaCl and 1-50 weight % lactose, between 60-99 weight % NaCl and 1-40 weight % lactose, or between 70-99 weight % NaCl and 1-30 weight % lactose, such as preferably between 75-99 weight % NaCl and 1-25 weight % lactose, more preferably between 80-99 weight % NaCl and 1-20 weight % lactose, more preferably between 85-99 weight % NaCl and 1-25 weight % lactose, more preferably between 90-99 weight % NaCl and 1-10 weight % lactose; most preferably the dry powder composition comprises approximately 95 weight % NaCl and 5 weight % lactose.

In one embodiment, the composition consists essentially of NaCl and lactose, and the weight ratio is as disclosed above.

In preferred embodiments, the dry powder composition of the present invention substantially consists of NaCl and lactose in a ratio of approximately 95:5 based on weight. “Substantially consists of” and “consists essentially of” means that only minor impurities may be present in the composition which are considered inert in regards to having a therapeutic effect and also considered inert in regard to functioning as an anti-agglomerating agent.

As further disclosed herein and evident from the examples provided here, when administered to a mammalian subject, the NaCl in the dry powder composition acts as a therapeutic agent. In one embodiment, NaCl is the sole therapeutic agent of the pharmaceutical composition, with all other components being inert in regards to the therapeutic effect of treating, and/or preventing and/or ameliorating viral infection in airways of a mammal. In one embodiment, the dry powder composition comprises lactose, and the sole therapeutic agents of the composition are NaCl and lactose. In another embodiment, additional therapeutic agents may be present in the composition, such as other antiviral drugs, such as oseltamivir, zanamavir, amantadine, rimantadine, ribavirin, gancyclovir, valgancyclovir, foscavir, Cytogam® (Cytomegalovirus Immune Globulin), pleconaril, rupintrivir, palivizumab, motavizumab, cytarabine, docosanol, denotivir, cidofovir, acyclovir, or such as Paxlovid™ (nirmatrelvir and ritonavir) or Lagevrio™ (molnupiravir).

### I.ii Particle Size

Dry powder formulations are prepared with the appropriate particle diameter and density for localized delivery to selected regions of the respiratory tract. For example, higher density and/or larger particles may be used for upper airway delivery, while lower density and/or smaller particles may be used for lower airway delivery. Similarly, a mixture of different sized particles can be administered to target different regions of the lung in one administration.

For the present invention, delivery to the respirator tract and airway is preferred, hence the dry composition of the present invention is preferably micronised to a respirable particle size range of 1-10 μm

The dry powder compositions is administered by dry inhalation as an aerosol—i.e. a fine this cloud of dry particles. Dry powder aerosols for inhalation therapy are preferably produced with mean diameters in the range between 1-10 microns. Preferably the volume median geometric diameter for the aerosol particles is less than about 10 microns.

The preferred volume median geometric diameter for aerosol particles is about 5 microns. For example, the aerosol can contain particles that have a volume median geometric diameter between about 0.1 and about 30 microns, between about 0.5 and about 20 microns, between about 0.5 and about 10 microns, between about 1.0 and about 5.0 microns, or between about 2.0 and 5.0 microns.

It is preferred that all the dry powder particles have a particle size (i.e. mean diameter) less than 20, 19, 18, 17, 16, 15, 14, 13, 12, or 11 microns, preferably less than 10, 9, 8, 7, or 6 microns, more preferably all the dry particles have a particle size around 5 microns.

The Malvern particle size analyzer is used to define and measure the particle size distribution, such as the Malvern Mastersizer 3000 device. The particle-size distribution (PSD) of the powder defines the relative amount of particles present according to size.

In one embodiment, the dry powder composition has a particle size distribution of at least 50% of the particles being in the range 1-10 μm. In a preferred embodiment, the dry powder composition has a particle size distribution of at least 60% of the particles being in the range 1-10 μm. In a more preferred embodiment, the dry powder composition has a particle size distribution of at least 70% of the particles being in the range 1-10 μm. In a most preferred embodiment, the dry powder composition has a particle size distribution of at least 80% of the particles being in the range 1-10 μm. In a still further preferred embodiment, the dry powder composition has a particle size distribution of at least 90% of the particles being in the range 1-10 μm, more preferably at least 95% of the particles being in the range 1-10 μm.

In one embodiment, the dry powder composition has a particle size distribution of at least 50% of the particles being in the range 2-5 μm. In a preferred embodiment, the dry powder composition has a particle size distribution of at least 60% of the particles being in the range 2-5 μm. In a more preferred embodiment, the dry powder composition has a particle size distribution of at least 70% of the particles being in the range 2-5 μm. In a most preferred embodiment, the dry powder composition has a particle size distribution of at least 80% of the particles being in the range 2-5 μm. In a still further preferred embodiment, the dry powder composition has a particle size distribution of at least 90% of the particles being in the range 2-5 μm, more preferably at least 95% of the particles being in the range 2-5 μm.

### I.iii Preparation of the Pharmaceutical Dry Powder Formulation

Generally, pharmaceutical formulations that are dry powders may be produced by any of spray drying, freeze drying, jet milling, single and double emulsion solvent evaporation, and super-critical fluids. Preferably, dry powder formulations are produced by jet milling. Jet milling is a particle size reduction method in which un-milled powder(s) are fed into a milling chamber. Inside the chamber compressed air and/or nitrogen, usually in a vortex motion, promotes particle-to-particle collisions. Typically jet mills are designed to output particles below a certain size while continuing to mill particles above that size, resulting in a narrow size distribution of the resulting product. Jet milled powders that contain salt according to the present invention, such as sodium salt, can be readily prepared using conventional methods.

Dry powder formulations can also be prepared by blending individual components into the final pharmaceutical formulation. For example, a first dry powder that contains a salt can be blended with additional dry powders that contain excipients (e.g., lactose) to be included in the blend. The blend can contain any desired relative amounts or ratios of salt, excipients and optionally other additional ingredients, according to the teachings of the present invention.

II. a Pharmaceutical Dry Powder Composition for Use in Treatment and/or Prevention of Viral Respiratory Tract Infection

An aspect of the invention provides a pharmaceutical composition as described herein for use as a medicament, wherein said pharmaceutical composition is administered by dry powder inhalation to a mammal in need thereof. The mammal may be a human, a primate, a mouse, a rat, a dog, a cat, a horse, as well as livestock or animals grown for food consumption, e.g., cattle, sheep, pigs, chickens, and goats. In a preferred embodiment, the mammal is a human.

As demonstrated herein, a pharmaceutical composition of the invention is capable of inhibiting viral infection, such as attenuating the viral load of SARS-COV-2 (Example 1). As further evidenced herein, administration of the pharmaceutical composition to the respiratory tract of a mammal suffering from a viral infection, such as COVID, facilitates a significant reduction in cough symptoms in the patient (Example 3).

The invention provides a pharmaceutical composition as described herein for use in treating and/or preventing infectious diseases of the respiratory tract, preferably viral infections of the respiratory tract, wherein said composition is administered by dry powder inhalation.

The invention further provides methods for treatment (including prophylactic treatment) of infectious diseases of the respiratory tract, such as viral infections of the respiratory tract. In one embodiment, the invention provides a method for treating (including prophylactically treating) an individual with a viral infection of the respiratory tract, an individual exhibiting symptoms of a viral infection of the respiratory tract, or an individual at risk of contracting a viral infection of the respiratory tract, comprising administering to the respiratory tract of the individual an effective amount of a pharmaceutical formulation as described herein.

In certain embodiments, the viral infection is caused by a virus selected from the group consisting of influenza virus (e.g., Influenza virus A, Influenza virus B), respiratory syncytial virus, adenovirus, metapneumovirus, cytomegalovirus, parainfluenza virus (e.g., hPIV-1, hPIV-2, hPIV-3, hPIV-4), rhinovirus, adenovirus, coxsackie virus, echo virus, herpes simplex virus, poxvirus (e.g. smallpox), enterovirus, and corona virus. In preferred embodiments, the viral infection is a corona virus, such as a SARS-coronavirus. In further preferred embodiments, the viral infection is of the genus Betacoronavirus, such as the subgenus Sarbecovirus (SARS Betacoronavirus). In a most preferred embodiment, the viral infection is specifically SARS-COV-2.

In a preferred embodiment, the invention provides a pharmaceutical composition as described herein for use in treating and/or preventing a viral respiratory tract infection caused by coronavirus, wherein said composition is administered to a mammalian subject by dry powder inhalation. In a preferred embodiment, the invention provides a pharmaceutical composition as described herein for use in treating and/or preventing a viral respiratory tract infection caused by SARS-COV-2, wherein said composition is administered to a mammalian subject by dry powder inhalation.

In one embodiment, the invention provides a pharmaceutical composition as described herein for use in treating and/or preventing infectious diseases of the respiratory tract, preferably viral infections of the respiratory tract, wherein said composition is administered by dry powder inhalation, and wherein the composition reduces symptoms of the viral respiratory tract infection. In some embodiments, the symptoms may be selected form one or more of fever, shortness of breath, nasal blockage, cough (e.g. dry cough), sore throat, runny nose, vomiting, nausea, diarrhoea, sneezing, fatigue, body ache, myalgia, dysgeusia, anosmia, headache, and chest pain.

In a preferred embodiment, the pharmaceutical composition of the present invention reduces cough, such as dry cough, caused by viral respiratory tract infection. In a preferred embodiment, the pharmaceutical composition of the present invention reduces the frequency of cough caused by viral respiratory tract infection, as compared to a person not treated with the composition. In one embodiment, at least 10, 15, 20, 25, 30, 35, 40, 45, 50, 55, 60, 65, 70, 75, 80, 85 or at least 95% reduction in frequency of cough is achieved after 5 days of treatment, as compared to an untreated patient. In one embodiment, at least 10, 15, 20, 25, 30, 35, 40, 45, 50, 55, 60, 65, 70, 75, 80, 85 or at least 95% reduction in frequency of cough is achieved after 10 days of treatment, as compared to an untreated patient. Cough frequency is typically measured by patient self-assessment, such as by the St. George's Respiratory Questionnaire (SGRQ) by

American Toracic Scociety. This is a disease-specific instrument designed to measure impact on overall health, daily life, and perceived well-being in patients with obstructive airways disease. (www.thoracic.org/members/assemblies/assemblies/srn/questionaires/sgrq.php)

In a preferred embodiment, the pharmaceutical composition of the present invention reduces the severity of cough, such as dry cough, caused by viral respiratory tract infection, as compared to a person not treated with the composition. In one embodiment, at least 10, 15, 20, 25, 30, 35, 40, 45, 50, 55, 60, 65, 70, 75, 80, 85 or at least 95% reduction in severity of cough is achieved after 5 days of treatment, as compared to an untreated patient. In one embodiment, at least 10, 15, 20, 25, 30, 35, 40, 45, 50, 55, 60, 65, 70, 75, 80, 85 or at least 95% reduction in severity of cough is achieved after 10 days of treatment, as compared to an untreated patient. Cough severity is typically measured by patient self-assessment, such as by the St. George's Respiratory Questionnaire.

In one embodiment, use of a composition according to the present invention facilities a significant reduction in recovery time in a patient suffering from a viral infection, compared to if the same patient had not been treated using such composition. In one embodiment, the dry powder composition of the present invention reduces the time for resolution of one or more symptoms caused by the viral respiratory tract infection. In one embodiment, the average recovery time from cough (e.g. as a symptom of Covid-19 affecting the airways) is significantly improved. The terms “recovery time” and “time for resolution” are used herein synonymously. In one embodiment, an average of 30, 32, 34, 36, 38, 40, 42, 44, 46, 48, 50, 51, 52, 53, 54, 55, 56, 57, 58, 59, 60, 61, 62, 63, 64, 65, 66, 67, 68, 69 or even 70% of patients suffering from a viral respiratory tract infection, treated with the dry powder composition of the present invention, recover from cough within 10 days of onset. In one embodiment, an average of 30, 32, 34, 36, 38, 40, 42, 44, 46, 48, 50, 51, 52, 53, 54, 55, 56, 57, 58, 59, 60, 61, 62, 63, 64, 65, 66, 67, 68, 69 or even 70% of corona virus patients treated with the dry powder composition of the present invention recover from cough within 10 days of onset. The term “recover from cough” covers complete cessation of cough in a patient or reduction of cough to a patient's normal cough level when the patient does not suffer from said viral respiratory tract infection.

In one embodiment, an average of 60, 62, 64, 66, 68, 70, 72, 74, 75, 76, 77, 78, 79, 80, 81, 82, 83, 84, 85, 86, 87, 88, 89 or even by 90% of patients suffering from a viral respiratory tract infection, treated with the dry powder composition of the present invention recover from cough within 18 days of onset. In one embodiment, an average of 60, 62, 64, 66, 68, 70, 72, 74, 75, 76, 77, 78, 79, 80, 81, 82, 83, 84, 85, 86, 87, 88, 89 or even by 90% of corona virus patients treated with the dry powder composition of the present invention recover from cough within 18 days of onset.

As demonstrated herein (Example 2), on average, corona virus patients treated with a dry powder composition of the present invention has 30-40% faster recovery of COVID-19-induced cough than a control group not receiving the treatment.

In one embodiment, the present invention provides a dry powder composition as disclosed herein for use in treatment of one or more symptoms of the viral respiratory tract infection, wherein the time for resolution of the one or more symptoms in a patient is on average improved by at least 10, 12, 14, 16, 18, or 20% compared to a control group not receiving the treatment, such as improved by at least 22, 24, 26, 28, or 30% compared to the control group not receiving the treatment, such as improved by at least 32, 34, 36, 38, or 40% compared to the control group not receiving the treatment, such as improved by at least 42, 44, 46, 48, or 50% compared to the control group not receiving the treatment.

In one embodiment, the present invention provides a dry powder composition as disclosed herein for use in treatment of cough, such as dry cough, caused by a viral respiratory tract infection, wherein time for resolution of the cough in a patient suffering from said viral respiratory tract infection is on average improved by at least 10, 12, 14, 16, 18, or 20% compared to a control group not receiving the treatment, such as improved by at least 22, 24, 26, 28, or 30% compared to the control group not receiving the treatment, such as improved by at least 32, 34, 36, 38, or 40% compared to the control group not receiving the treatment, such as improved by at least 42, 44, 46, 48, or 50% compared to the control group not receiving the treatment. In a preferred embodiment, the cough in said patient is caused by coronavirus.

In a further aspect, the invention provides a pharmaceutical composition as described herein for use in treating and/or preventing a pulmonary disease (i.e. a type of disease that affects the lungs and other parts of the respiratory system), wherein said composition is administered by dry powder inhalation.

In one embodiment, the invention provides a method for treating (including prophylactically treating) an individual with a pulmonary disease-such as an individual having a pulmonary disease, exhibiting symptoms of a pulmonary disease, or susceptible to a pulmonary disease-comprising administering to the respiratory tract of the individual an effective amount of a pharmaceutical formulation by dry powder inhalation, according to the present invention.

Preferably, the invention provides a dry powder composition as disclosed herein for use in preventing and/or treating a viral respiratory tract infection caused by coronavirus, such as SARS-COV-2, wherein said composition is administered to a mammalian subject by dry powder inhalation. Preferably the dry powder composition comprises at least 80, 85, or 90 weight % sodium chloride, or more preferably around 95 weight % sodium chloride. The pharmaceutical formulation used for treating (including prophylactically treating) a respiratory tract infection preferably comprises a sodium chloride salt and lactose, preferably in a ratio of NaCl salt to lactose of about 95:5 (wt/wt). Further preferably, the invention provides a dry powder composition as disclosed herein for use in treating and/or ameliorating cough caused by viral respiratory tract infection, wherein said composition is administered to a mammalian subject by dry powder inhalation. Preferably the dry powder composition comprises at least 80, 85, or 90 weight % sodium chloride, or more preferably around 95 weight % sodium chloride. The pharmaceutical formulation used for treating and/or ameliorating cough preferably comprises a sodium chloride salt and lactose, preferably in a ratio of NaCl salt to lactose of about 95:5 (wt/wt).

### III Administration of the Pharmaceutical Dry Powder Formulation

It is an essential feature of the invention that the dry powder composition is administered by dry powder inhalation. The present invention thereby provides a NaCl particle inhalation therapy which has a huge advantage over the use of standard nebulized solutions, both for patients and healthcare workers, considering the safety aspect of minimized viral contamination of the aerosols.

### III.i Dry Powder Inhalation

The geometry of the airways is an important consideration when selecting a suitable method for producing and delivering aerosols of pharmaceutical formulations to the respiratory tract. The lungs are designed to entrap particles of foreign matter that are breathed in, such as dust. As disclosed herein, for administration, dry powder inhalation with the appropriate particle size is selected for preferential delivery to the desired region of the respiratory tract. Particles between 0.6-5 microns in diameter generally reach the deep lungs, while particles about 3 microns or larger diameter generally stay in the upper airway.

Broad clinical application of dry powder inhalation delivery has been limited by difficulties in generating dry powders of appropriate particle size, particle density, and dispersibility, in keeping the dry powder stored in a dry state, and in developing a convenient, hand-held device that effectively disperses the respirable dry particles to be inhaled in air.

The pharmaceutical formulations as described herein are intended for administration to the respiratory tract (e.g., to the mucosal surface of the respiratory tract) in dry powder form. Preferably, a pharmaceutical formulation as described herein is aerosolized for administration. Many suitable methods and devices that are conventional and wellknown in the art can be used to dry powder aerosolize the formulation, such as a dry power inhaler.

### III.ii Dry Powder Inhaler

Inhalable dry powder is typically administered to the respiratory tract including the lungs and the nasal membranes via a dry powder inhaler.

In one embodiment the dry powder inhaler is suitable for nasal inhalation. In another embodiment the dry powder inhaler is suitable for oral inhalation. In yet another embodiment, the dry powder composition is orally and nasally inhalable. Several suitable dry inhaler devices exist. The composition is preferably administered in a single, breath-activated step using a breath-activated dry powder inhaler (DPI).

In a particular embodiment the dry powder inhaler is as described in WO2015004227 (U.S. Pat. No. 10,583,261 B2), the entireties of which are incorporated by reference herein.

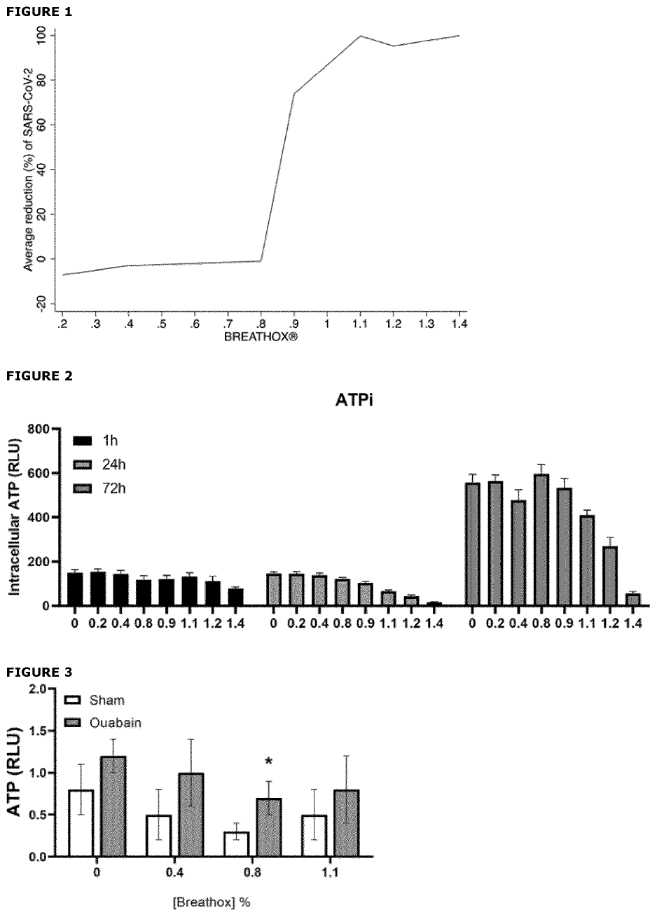

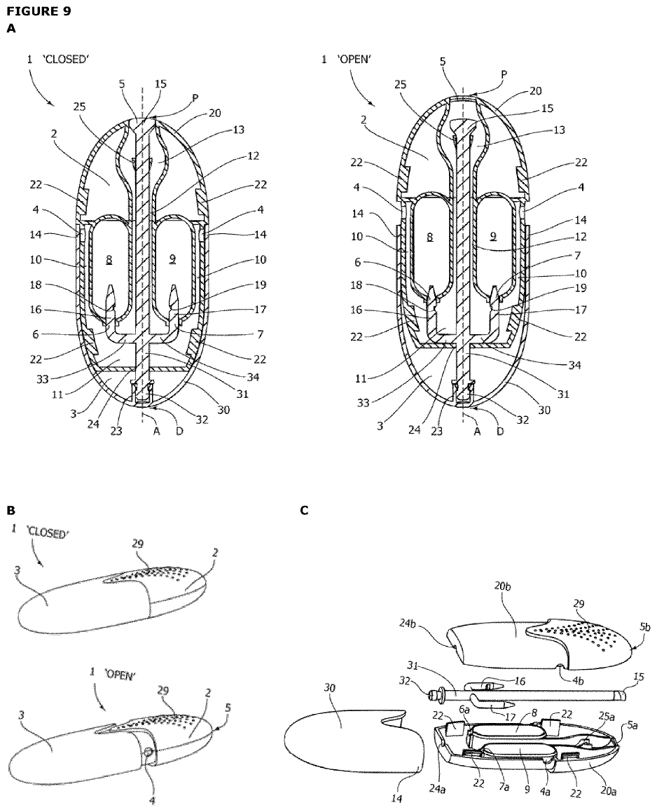

In particular, the dry inhaler as shown in FIG. 9 (copies of FIGS. 1a, 1b, 2a, 2b, and 3 in WO2015004227) is a preferred embodiment. Hence, in a preferred embodiment of the present invention, the dry powder inhaler used for administering the dry powder composition of the present invention has in an axial direction a proximal end (P) for insertion into the mouth of a user, and a distal end (D) opposite to the proximal end (P), wherein the inhaler comprises an inlet, an outlet arranged at the proximal end (P), an air passage extending from the inlet to the outlet, and a reservoir communicating with the air passage through a release orifice, the reservoir containing a dispersible substance, wherein the inhaler has a proximal part comprising the outlet, and a distal part attached to the proximal part, wherein the proximal part is linearly slidable along the axial direction (A) with respect to the distal part between an ‘OPEN’ position where the proximal part is deployed from the distal part, and a ‘CLOSED’ position where the proximal part is retracted towards the distal part, wherein the inhaler further comprises an inlet valve member, an outlet valve member, and a reservoir valve member, said inlet, outlet and reservoir valve members being arranged so as to simultaneously close the inlet, the outlet, and the release orifice when the proximal part is brought from the ‘OPEN’ position into the ‘CLOSED’ position, and to simultaneously open the inlet, open the outlet, and deliver an amount of the dispersible substance from the reservoir through the release orifice to the air passage when the proximal part is brought from the ‘CLOSED’ position into the ‘OPEN’ position.

In a further preferred embodiment, the dry powder inhaler is an inhaler (1) having in an axial direction a proximal end (P) for insertion into the mouth of a user, and a distal 40 end (D) opposite to the proximal end (P), wherein the inhaler (1) comprises an inlet (4), an outlet (5) arranged at the proximal end (P), an air passage (10, 11, 12, 13) extending from the inlet (4) to the outlet (5), and a reservoir (8, 9) communicating with the air passage (10, 11, 12, 13) through a release orifice (6, 7), the reservoir (8, 9) containing a dispersible substance, wherein the inhaler (1) has a proximal part (2) comprising the outlet (5), and a distal part (3) attached to the proximal part (2), wherein the proximal part (2) is linearly slidable along the axial direction (A) with respect to the distal part (3) between an ‘OPEN’ position where the proximal part (2) is deployed from the distal part (3), and a ‘CLOSED’ position where the proximal part (2) is retracted towards the distal part (3), wherein the inhaler (1) further comprises an inlet valve member (14), and an outlet valve member (15);
wherein the reservoir (8, 9) is arranged in the proximal part (2);
wherein the inlet (4) comprises one or more openings in a peripheral housing wall (20) of the proximal part (2), the one or more openings facing radially outward, away from the axial direction (A), and the inlet valve member (14) is formed by a peripheral housing wall (30) of the distal part (3) covering the openings when the proximal part (2) is in the ‘CLOSED’ position;
wherein the outlet (5) comprises an axially oriented aperture, wherein the outlet valve member (15) is formed as a plug attached to the distal part (3), the plug blocking the outlet (5) when the proximal part (2) is in the ‘CLOSED’ position;
wherein the release orifice (6, 7) is oriented in the axial direction (A); and
wherein the inhaler (1) further comprises a reservoir valve member (16, 17), wherein the reservoir valve member (16, 17) is formed as a peg travelling in the axial direction (A), the peg being fixed to the distal part (3) via an axially extending stem, wherein the peg blocks the release orifice (6, 7) when the proximal part (2) is in the ‘CLOSED’ position;

whereby said inlet valve member (14), outlet valve member (15) and reservoir valve member (16, 17) are arranged so as
to simultaneously close the inlet (4), the outlet (5), and the release orifice (6, 7) when the proximal part (2) is retracted towards the distal part along the axial direction from the ‘OPEN’ position into the ‘CLOSED’ position, and
to simultaneously open the inlet (4), open the outlet (5), and deliver an amount of the dispersible substance from the reservoir (8, 9) through the release orifice (6, 7) to the air passage (10, 11, 12, 13) when the proximal part (2) is deployed from the distal part along the axial direction from the ‘CLOSED’ position into the ‘OPEN’ position.

Further preferred embodiments are specifically described in WO2015004227. In one preferred embodiment, the dry powder inhaler for administering of the dry powder composition of the present invention is BREATHOX®.

The BREATHOX® inhaler contains 1000 mg+150 mg dry powder, referred to herein as BREATHOX® POWDER. The device delivers 90% of doses within 2.1±0.6 mg per inhalation and 95% of doses within the specified critical level of 2.0±1.0 mg. This equals a minimum of 300 inhalations per device. The lifetime of 300 doses is based upon the worst-case scenario, being 90% of doses of 2.7 mg (2.1±0.6 mg). 2.7 mg times 300 doses equal 810 mg of salt, which is 40 mg lower than the minimum filling tolerance (850 mg). 40 mg is left in the device; the probability of having maximum dispensing throughout the lifetime is significantly low.

### III.iii Dosage Regime

The pharmaceutical formulation of the present invention can be delivered to the upper respiratory tract (e.g., nasal passages, nasal cavity, throat, pharynx), respiratory airways (e.g., larynx, tranchea, bronchi, bronchioles) and/or lungs (e.g., respiratory bronchioles, alveolar ducts, alveolar sacs, alveoli).

In one aspect, the present invention relates to a dry powder composition as disclosed herein, for use in preventing or reducing viral growth and inhibiting viral replication in a respiratory tract of a mammal, such as a human, wherein an effective amount of the dry powder composition is administered to the mammal by dry powder inhalation.

In a further aspect, the present invention relates to a method for preventing or reducing viral growth and inhibiting viral replication in a respiratory tract of a mammal, such as a human, wherein an effective amount the dry powder composition of the present invention is administered to a mammal in need of said treatment by dry powder inhalation.

An effective amount of a pharmaceutical formulation as described herein is administered to an individual in need thereof, such as an individual who has a respiratory tract infection, who is exhibiting symptoms of a respiratory tract infection, or who is at risk of contracting a respiratory tract infection. An “effective amount” is an amount that is sufficient to achieve the desired therapeutic or prophylactic effect, such as an amount sufficient to reduce symptoms of infection.

Preferably, inhalation devices should be able to deliver a therapeutically effective amount of a composition described herein in a single inhalation. In some cases however, to achieve the intended therapeutic results, multiple inhalations and/or frequent administration may be required.

Generally, a pharmaceutical formulation is administered once, twice, three, four, five, six or more times a day, as needed. Suitable intervals between doses that provide the desired therapeutic effect may be determined based on the severity of the condition (e.g. infection).

The composition of the present invention is non-toxic to a human subject, and most mammals. Dosing may be based on the desired amount of salt to be delivered to the respiratory tract. The daily dosage administered may depend on the concentration of viral particles in the airways and the frequency of such viral particles as inhaled.

During the covid-trial (Example 3), patients from group 1 received 5 sessions of BREATHOX® a day, equivalent to 22.5 to 30 mg BREATHOX® POWDER (NaCl powder); while patients from group 2 instead received 10 sessions of BREATHOX® a day, equivalent to 45 to 60 mg BREATHOX® POWDER (NaCl powder).

In a preferred embodiment, the total daily dosage administered is from 4.5 to 60 mg, corresponding to 1 to 10 sessions per day, such as when using the BREATHOX® device.

In one embodiment, the total daily dosage of the composition of the present invention administered to a human subject is between 0.5-200 mg, between 1-180 mg, between 2-150 mg, between 3-120, between 4-90 mg, or preferably between 5-60 mg, such as between 10-55 mg, between 20-50 mg, between 30-45 mg, such as approximately 40 mg; such as between 5-55 mg, between 5-50 mg, between 5-45 mg, between 5-40 mg, between 5-35 mg, between 5-30 mg, between 5-25 mg, between 5-20 mg, between 5-15 mg, or between 5-10 mg; such as between 55-60 mg, between 50-60 mg, between 45-60 mg, between 40-60 mg, between 35-60 mg, between 30-60 mg, between 25-60 mg, between 20-60 mg, between 15-60 mg, or between 10-60 mg. These daily dosages are particularly preferred for treatment and/or prevention of corona virus, such as treatment and/or prevention of COVID-19.

In one embodiment, the total daily dosage of NaCl administered to a human subject is between 0.5-200 mg, between 1-180 mg, between 2-150 mg, between 3-120 mg, between 4-90 mg, or preferably between 5-60 mg, such as between 10-55 mg, between 20-50 mg, between 30-45 mg, such as approximately 40 mg; such as between 5-55 mg, between 5-50 mg, between 5-45 mg, between 5-40 mg, between 5-35 mg, between 5-30 mg, between 5-25 mg, between 5-20 mg, between 5-15 mg, or between 5-10 mg; such as between 55-60 mg, between 50-60 mg, between 45-60 mg, between 40-60 mg, between 35-60 mg, between 30-60 mg, between 25-60 mg, between 20-60 mg, between 15-60 mg, or between 10-60 mg. These daily dosages are particularly preferred for treatment and/or prevention of corona virus, such as treatment and/or prevention of COVID-19.

In a preferred embodiment, the total daily dosage of the composition of the present invention administered to a human subject is from 0.005-5 mg/kg body weight, from 0.005-4 mg/kg body weight, from 0.005-3 mg/kg body weight, from 0.005-2 mg/kg body weight, from 0.005-1 mg/kg body weight, from 0.005-0.5 mg/kg body weight, from 0.005-0.2 mg/kg body weight, from 0.005-0.1 mg/kg body weight, or from 0.005-0.05 mg/kg body weight, such as from 4-5 mg/kg body weight, from 3-5 mg/kg body weight, from 2-5 mg/kg body weight, from 1-5 mg/kg body weight, from 0.5-5 mg/kg body weight, from 0.2-5 mg/kg body weight, from 0.1-5 mg/kg body weight, or from 0.05-5 mg/kg body weight, such as from 0.05-4 mg/kg body weight, from 0.1-3 mg/kg body weight, from 0.2-2 mg/kg body weight, or from 0.5-1 mg/kg body weight.

In a preferred embodiment, the total daily dosage of NaCl administered to a human subject is from 0.005-5 mg/kg body weight, from 0.005-4 mg/kg body weight, from 0.005-3 mg/kg body weight, from 0.005-2 mg/kg body weight, from 0.005-1 mg/kg body weight, from 0.005-0.5 mg/kg body weight, from 0.005-0.2 mg/kg body weight, from 0.005-0.1 mg/kg body weight, or from 0.005-0.05 mg/kg body weight, such as from 4-5 mg/kg body weight, from 3-5 mg/kg body weight, from 2-5 mg/kg body weight, from 1-5 mg/kg body weight, from 0.5-5 mg/kg body weight, from 0.2-5 mg/kg body weight, from 0.1-5 mg/kg body weight, or from 0.05-5 mg/kg body weight, such as from 0.05-4 mg/kg body weight, from 0.1-3 mg/kg body weight, from 0.2-2 mg/kg body weight, or from 0.5-1 mg/kg body weight.

In one embodiment, the dry powder composition is administered to the patient once a day. In preferred embodiments, the dry powder composition of the present invention is administered to the patient several times a day, such as 2, 3, 4, 5, 6, 7, 8, 9, 10 or more times each day, such that the total daily dosage is spread out over several unit doses. This may also be referred to as administered in several sessions throughout the day. In one embodiment, the dry powder composition is administered to the patient 1-10 times each day, 1-9 times each day, 1-8 times each day, 1-7 times each day, 1-6 times each day, 1-5 times each day, 1-4 times each day, 1-3 times each day, or 1-2 times each day. In one embodiment, the dry powder composition is administered to the patient 2-10 times each day, 3-10 times each day, 4-10 times each day, 5-10 times each day, 6-10 times each day, 7-10 times each day, 8-10 times each day, or 9-10 times each day. In one embodiment, the dry powder composition is administered to the patient preferably 1-5 times each day, such as 2-5 times each day, such as 2-4 times each day, such as 3 times each day.

Whenever a mammal, such as a human, inhales one dose of the dry powder composition, such dose is considered a unit dose. The unit dose will depend on the type of dry powder inhaler used. One administering session may comprise multiples inhalations—i.e. multiple dose units, such as 2, 3, 4, 5, 6 or more dose units per session. Typically a dry powder inhaler is set so that the amount inhaled in one inhalation is from 0.25 to 25 mg, such as from 0.25 to 4 mg. In a most preferred embodiment, the inhaler delivers 1.5 to 2 mg dry powder in one inhalation.

As an illustrative example, and preferred embodiment, the BREATHOX® inhaler contains 1000 mg±150 mg BREATHOX® POWDER (a NaCl powder according to the present invention). The device delivers 90% of doses within 2.1±0.6 mg per inhalation and 95% of doses within the specified critical level of 2.0±1.0 mg. This equals a minimum of 300 inhalations per device. The lifetime of 300 doses is based upon the worst-case scenario, being 90% of doses of 2.7 mg (2.1+0.6 mg). 2.7 mg times 300 doses equal 810 mg of salt, which is 40 mg lower than the minimum filling tolerance (850 mg). 40 mg is left in the device; the probability of having maximum dispensing throughout the lifetime is significantly low.

For COVID-19 disease, the optimal and recommended dose was found to be 4 inhalations (2 nasaly, 2 orally) five times daily until symptoms are resolved (see Example 3). This is equivalent to approximately 40 mg of NaCl powder. Users can also inhale as needed (ten sessions per day is the maximum recommended use, 1 session=3 inhalations). BREATHOX® should be inhaled every time a dose is released.

In a preferred embodiment, a unit dosage administered to a patient is from 0.25 to 25 mg, such as from 0.25 to 4 mg. In a most preferred embodiment, the unit dosage administered to a patient is from 1.5 to 2 mg dry powder.

In one embodiment, the pharmaceutical dry powder formulation of the present invention is administered by a dry powder inhaler to patients in amount of 0.001-0.5 mg dry powder/kg body weight/dose, such as 0.002-0.2 mg dry powder/kg body weight/dose, preferably 0.01-0.04 mg dry powder/kg body weight/dose.

In further embodiments the dry powder composition is administered successive or simultaneous with one or more other medicine(s) or drug(s). It is contemplated that the compositions of the present invention may be used in combination with or to enhance the activity of other antimicrobial agents, or to effectuate a synergism between the multiple agents such that the combination is more effective than the sum of the efficacy of either agent considered independently. Combinations with other agents may also be useful to allow such other agents to be used at lower doses, thereby reducing concerns over toxicity. The combination may inhibit microbe replication, reduce symptoms, shorten the duration of infection, and/or reduce microbe burden in the patient.

In some embodiments, the dry powder composition of the present invention is administered together with a different drug to treat, ameliorate and/or prevent the same disease, disorder or condition. In other embodiments, dry powder composition is used in combination with a different drug to treat, ameliorate and/or prevent a comorbidity.

In some embodiments, the combination therapy involves administering both agents/therapies at the same time. This may be achieved by administering a single composition or pharmacological formulation that includes both agents, or by administering two distinct compositions or formulations at the same time, where each composition contains one agent. Alternatively, the treatment using the dry powder composition of the present invention may precede or follow the “other” treatment by intervals ranging from minutes to weeks.

In one embodiment, the present invention envisages the use of one or more traditional antiviral therapies in combination with the dry powder composition of the present invention. In another embodiment, the dry powder composition is administered together with and/or before and/or after treatment with an antiviral drug. In a further embodiment the dry powder composition is administered prior to, successive or simultaneous with a lung medicine, such as inhaled corticosteroid.

### III.iv Reducing Transmission or Spread

Use of dry powder formulations, administered by dry powder inhalation reduces transmission and/or spread of viral infection, compared to e.g. the use of a nebuliser.

In a further aspect, the invention provides methods for reducing transmission or spread of a viral respiratory tract infection, comprising administering to the respiratory tract (e.g., lungs, nasal cavity) of an individual infected with a virus that causes a respiratory tract infection, exhibiting symptoms of a respiratory tract infection, or at risk of contracting a respiratory tract infection by a virus, an effective amount of a pharmaceutical formulation as described herein.

### EXAMPLES

The present invention is illustrated by the following examples that, however, are not to be construed as limiting the scope of protection. The features disclosed in the foregoing description and in the following examples may, both separately and in any combination, be material for realizing the invention in diverse forms thereof.

BREATHOX® is a dry powder inhaler. It is a substance-based, non-sterile, breath-actuated dry particle inhaler that consists of 5 injection-molded acrylonitrile butadiene styrene (ABS) plastic polymer parts assembled into one unit. The device is disclosed in WO2015004227A1.

The BREATHOX® device contains BREATHOX® POWDER, which comprises 95 wt % NaCl and 5 wt % lactose. The NaCl is obtained from Hvornum Salt Diapir, Denmark. The NaCl is mixed and micronised with alpha-lactose monohydrate. The NaCl: lactose ratio of the powder is 95%: 5% (on weight basis), with #2% tolerance. This is also referred to as the ‘NaCl powder’.

NaCl is of pharmaceutical grade, manufactured in compliance with the Monograph Sodium Chloride's current version no. 193 of the European Pharmacopoeia. Alpha-lactose monohydrate of pharmaceutical grade and conforms with Ph. Eur.

The BREATHOX® POWDER is micronised to a respirable particle size range of 1-10 μm and is considered a medicinal substance.

One inhalation (i.e. one unit dose) delivers approximately 1.5-2 mg BREATHOX® POWDER to the test subject.

### Example 1: Anti-Viral Effects of BREATHOX®—In Vitro Study Design

The antiviral effects of the dry powder composition (comprising 95 wt % NaCl and 5 wt % lactose) comprised in the BREATHOX® devise—i.e. the BREATHOX® POWDER, in attenuating the viral load of SARS-COV-2 was investigated by a pre-clinical, in vitro study carried out at a NB-3 Laboratory (Biosafety Level 3), following all the WHO Biosafety regulations and in compliance with Good Laboratory Practices (GLP).

Methodology: To analyse the antiviral activity of BREATHOX® there were three steps: First prepare the cells, virus and BREATHOX® dilutions. Then infect the cells and add BREATHOX® POWDER at different time-of-addition. Finally nucleic acid extraction and quantitative real time qPCR.

Vero cells (cell lineage derived from kidney epithelial cells extracted from an African green monkey; ATCC CCL-81-VHG) and Calu-3 cells (human non-small-cell lung cancer cell line that grows in adherent culture and displays epithelial morphology; ATCC Calu-3-HTB-55) were maintained in DMEM containing 0.6% NaCl, in a humidified atmosphere containing 5% CO₂ at 37° C. Virus titer was determined by plaque forming units per milliliter.

The BREATHOX® POWDER was dissolved in free-DMEM (Dulbecco's Modified Eagle's Medium). Seven concentrations of BREATHOX® POWDER in the cell medium were prepared: 0.2, 0.4, 0.8, 0.9, 1.1, 1.2, and 1.4% (weight/volume basis). The free-DMEM comprises 110 mM of NaCl, therefore the total NaCl concentration of the seven samples was 135, 160, 185, 210, 235, 260, and 285 mM, respectively.

For the anti-viral activity, there were 4 different time-of-addition experiments. Vero cells were seeded in a clear-bottomed 96-well plate (5×10⁴ cells/mL) and incubated for 24 h at 37° C. for cell adherence. They were then treated with increasing concentrations of the BREATHOX® POWDER at different stages of virus infection as described below. Four different BREATHOX® POWDER time-of-addition were evaluated, compromising virus pre-incubation (VPI), absorption (AD), post-infection (PI) and adsorption plus post-infection, named full-time (FT).

VPI: SARS-COV-2 variants were pre-incubated with increasing concentrations of BREATHOX® POWDER for 1 hour before infecting the cells. After adsorption, the inoculum was removed, replaced with media and maintained until the end of the experiment.

AD: the different concentration of BREATHOX® POWDER were added to the cell monolayer for 1 hour prior to virus infection and maintained during 1 hour for the viral attachment process. Then, the virus-BREATHOX® POWDER mixture was replaced with fresh DMEM until the end of the experiment.

PI: the virus was added to the cells to allow infection for 1 hour, and then virus-containing supernatant was replaced with different BREATHOX® POWDER concentration-containing medium until the end of the experiment.

FT: Vero cells were pretreated with different concentrations of BREATHOX® POWDER for 1 hour prior to virus infection, followed by incubation with virus for 1 hour in the presence of BREATHOX® POWDER.

Then, the virus mixture was removed, and cells were cultured with the same concentrations of BREATHOX® POWDER containing medium until the end of the experiment. For all experimental groups, cells were infected with virus at a multiplicity of infection (MOI) of 0.02, and at 72 h post-infection (hpi) the cell supernatants were collected for real time qPCR (RT-qPCR).

The supernatants collected after 72 h were submitted to Real-Time RT-PCR for detection and quantification of the SARS-COV-2 virus RNA. The extraction of total nucleic acid (RNA and DNA) was made using the semi-automated NucliSENS EASYMAG plataform (BioMerieux, Lyon, France), following the manufacturer's instructions. The quantification of viral RNA was done using the AgPath-ID One-Step RT-PCR-kit (Applied Biosystem, Weiterstadt, Germany), on an ABI 7500 SDS real-time PCR Machine (Applied Biosystems) using a reference published sequence of primers and probe for E gene (Corman et al., 2020). Numbers of RNA copies/mL were quantified using a specific in vitro-transcribed RNA quantification standard.

Findings: The RT-qPCR results were represented by RNA copies/mL and percentage of inhibition are represented for each treatment, in Table 1 (VPI), Table 2 (AD), Table 3 (PI) and Table 4 (FT).

TABLE 1
Quantification of SARS-CoV-2 viral RNA in the supernatant CCL-81 VERO cells
under VPI treatment with different concentrations of BREATHOX ®.

| | VIP Treatment triplicate | | | | | | |
|---|---|---|---|---|---|---|---|
| | A | | B | | C | | |
| | No. of | | No. of | | No. of | | Average |
| | RNA | Reduction | RNA | Reduction | RNA | Reduction | reduction |
| Breathox | copies | (%) | copies | (%) | copies | (%) | (%) |
| 0.2% | 2.76E+04 | −1.243.63 | 4.38E+05 | −21.252.37 | 7.65E+03 | −272.76 | −7.589.59 |
| 0.4% | 1.40E+04 | −582.05 | 7.01E+03 | −241.61 | 4.07E+05 | −19.732.44 | −6.852.03 |
| 0.8% | — | — | 2.66E+04 | −1.199.28 | — | — | −1.199.28 |
| 0.9% | — | — | 1.03E+05 | −4.908.51 | 1.40E+04 | −582.05 | −2.745.28 |
| 1.1% | 2.74E+04 | −1.234.64 | 3.83E+03 | −86.70 | 7.54E+03 | −267.79 | −529.71 |
| 1.2% | 6.64E+03 | −223.75 | 1.01E+05 | −4.841.71 | 2.83E+04 | −1.280.20 | −2.115.22 |
| 1.4% | 2.72E+04 | −1.225.71 | 1.02E+05 | −4.875.00 | 1.15E+05 | −5.513.98 | −3.871.56 |
| MOCK | 2.05E+03 | | | | | | |
| Inoculum | 4.94E+02 | | | | | | |

TABLE 2
Quantification of SARS-CoV-2 viral RNA in the supernatant CCL-81 VERO cells
under AD treatment with different concentrations of BREATHOX ®.

| | AD Treatment triplicate | | | | | | |
|---|---|---|---|---|---|---|---|
| | A | | B | | C | | |
| | No. of | | No. of | | No. of | | Average |
| | RNA | Reduction | RNA | Reduction | RNA | Reduction | reduction |
| Breathox | copies | (%) | copies | (%) | copies | (%) | (%) |
| 0.2% | 8.18E+05 | −42.819.34 | 8.18E+05 | −42.819.34 | 3.85E+05 | −20.135.90 | −35.258.19 |
| 0.4% | 7.34E+03 | −285.48 | 7.34E+03 | −285.48 | 1.45E+04 | −659.39 | −410.12 |
| 0.8% | 1.39E+04 | −629.41 | 1.39E+04 | −629.41 | 7.29E+03 | −282.90 | −513.91 |
| 0.9% | 7.39E+03 | −288.08 | 7.39E+03 | −288.08 | 2.87E+04 | −1.406.06 | −660.74 |
| 1.1% | 2.95E+04 | −1.447.05 | 2.95E+04 | −1.447.05 | 1.38E+04 | −624.53 | −1.172.88 |
| 1.2% | — | — | 3.99E+03 | −109.27 | 3.75E+03 | −97.00 | −103.13 |
| 1.4% | 2.76E+04 | −1.346.60 | 2.79E+04 | −1.366.16 | 3.68E+03 | −93.07 | −935.28 |
| MOCK | 1.90E+03 | | | | | | |
| Inoculum | 4.94E+02 | | | | | | |

TABLE 3
Quantification of SARS-CoV-2 viral RNA in the supernatant CCL-81 VERO cells
under PI treatment with different concentrations of BREATHOX ®.

| | PI Treatment triplicate | | | | | | |
|---|---|---|---|---|---|---|---|
| | A | | B | | C | | |
| | No. of | | No. of | | No. of | | Average |
| | RNA | Reduction | RNA | Reduction | RNA | Reduction | reduction |
| Breathox | copies | (%) | copies | (%) | copies | (%) | (%) |
| 0.2% | 1.93E+05 | 54.10 | 4.18E+05 | 0.67 | 2.05E+05 | 51.24 | 35.34 |
| 0.4% | 4.04E+05 | 3.95 | — | — | 7.96E+05 | −89.22 | −42.64 |
| 0.8% | 1.13E+05 | 73.17 | 1.14E+05 | 72.81 | 2.00E+05 | 52.53 | 66.17 |
| 0.9% | — | — | 5.14E+04 | 87.77 | 1.02E+05 | 75.74 | 81.76 |
| 1.1% | 7.80E+05 | −85.45 | 4.07E+05 | 3.30 | 2.01E+05 | 52.21 | −9.98 |
| 1.2% | 4.50E+05 | −6.94 | 3.99E+05 | 5.23 | — | — | −0.86 |
| 1.4% | 2.05E+05 | 51.24 | 4.04E+05 | 3.95 | — | — | 27.59 |
| MOCK | 4.21E+05 | | | | | | |
| Inoculum | 4.84E+02 | | | | | | |

TABLE 4
Quantification of SARS-CoV-2 viral RNA in the supernatant CCL-81 VERO cells
under FT treatment with different concentrations of BREATHOX ®.

| | FT Treatment triplicate | | | | | | |
|---|---|---|---|---|---|---|---|
| | A | | B | | C | | |
| | No. of | | No. of | | No. of | | Average |
| | RNA | Reduction | RNA | Reduction | RNA | Reduction | reduction |
| Breathox | copies | (%) | copies | (%) | copies | (%) | (%) |
| 0.2% | 8.74E+05 | −110.68 | 6.08E+06 | −1.366.16 | — | — | −738.42 |
| 0.4% | 2.99E+06 | −619.69 | 1.56E+06 | −275.27 | 1.59E+06 | −282.90 | −392.62 |
| 0.8% | — | — | 4.32E+05 | −4.11 | 4.04E+05 | 2.65 | −0.73 |
| 0.9% | 1.09E+05 | 73.71 | 1.08E+05 | 73.88 | — | — | 73.80 |
| 1.1% | 2.44E+02 | 100.00 | 1.87E+03 | 99.55 | 1.67E+01 | 100.00 | 99.85 |
| 1.2% | 2.97E+04 | 92.85 | 1.45E+04 | 96.51 | 1.36E+04 | 96.72 | 95.36 |
| 1.4% | 3.53E+01 | 100.00 | 9.23E+00 | 100.00 | 3.22E+01 | 100.00 | 100.00 |
| MOCK | 4.15E+05 | | | | | | |
| Inoculum | 4.84E+02 | | | | | | |

Based on the observation of cytopathic effect, and comparison of Real-Time RT-PCR results, it was found that none of the BREATHOX® POWDER concentrations in the VPI and AD treatments were able to reduce the SARS-COV-2 virus titer. However, the PI and FT treatments showed the ability to reduce viral load (See the FIG. 1 regarding the FT Treatment). The best results for the PI were at BREATHOX® POWDER concentrations of 0.8% and 0.9% for the PI treatment, with an average percentage of reduction of 66.17 and 81.76 respectively. However, there was a wide variation in the inhibition results of the PI treatment. Inhibition was not consistent, with higher concentrations of BREATHOX® POWDER showing lower percentages of inhibition as seen in Table 3. While for FT the best results were at BREATHOX® POWDER concentrations of 0.9, 1.1, 1.2, and 1.4%, with average percentages of reduction of 73.80; 99.85; 95.36 and 100.00 respectively. The FT treatment showed the best results in this experiment. With consistent viral reduction capacity, including inhibiting the visualization of the cytopathic effect of the 1.1% concentration of BREATHOX® POWDER up to 1.4% without causing any damage to the cell monolayer irreversibly.

Conclusion: Antiviral activities of BREATHOX® POWDER were confirmed by the in vitro study findings, which showed that BREATHOX® POWDER at concentrations of 0.9; 1.1; 1.2 and 1.4% (weight/volume basis) has an average virus inhibition of 73.80%, 99.85%, 95.36% and 100.00% respectively.

### Example 2: BREATHOX® Mechanism of Action

The mechanism of action of BREATHOX® POWDER in attenuating the viral load of SARS-CoV-2 was investigated. To elucidate the mode of action different assays were performed.

First the membrane potential variation was analyzed by microfluorimetry. Changes of the membrane potential of Vero cells exposed to increasing concentrations of NaCl was determined by plate microfluorimetry recordings with the FlexStation III microplate reader and the FLIPR Membrane Potential Assay Kit (Molecular Devices Corp., Sunnyvale, CA) following the manufacturer's instructions. Recordings of the fluorescence intensity of 5×10⁴ cells in black clear bottom of 96 wells plates were acquired at rest (point 0 h), 1 h, 24 h and 72 h after NaCl challenge (depolarizing agent).

Then to see the available ATP a luciferase assay was performed. Changes of the total concentration of ATP in Vero cells exposed to increasing concentrations of NaCl for 1 or 72 h was determined by plate microfluorimetry recordings with the FlexStation III microplate reader and the ATP Assay Kit (SIGMA-ALDRICH) following the manufacturer's instructions. The kit provides extremely sensitive results, detecting ATP release of 10-100 mammalian cells/well, based on the firefly luciferase-catalyzed oxidation of D-luciferin in the presence of ATP, which the amount of ATP is quantified by the amount of light (hv) produced. Recordings of the luminescence intensity of 5×10⁴ cells in black clear bottom of 96 wells plates were acquired at rest (point 0 h), 1 h, 24 h and 72 h after NaCl challenge (depolarizing agent).

In order to assess the effect of BREATHOX® POWDER on cellular ATP production the mitochondrial status was measured after every treatment condition (different BREATHOX® POWDER concentrations). For this purpose, the Vero cells were tested by the Seahorse assay that measures the mitochondrial metabolism. Intact Vero cells were assessed with a high-resolution respirometry assay. Two parameters were evaluated: oxygen consumption rate (OCR) and extracellular acidification rate (ECAR).

Mitochondrial respiration was evaluated at four different conditions: (i) basal respiration, corresponding to the cell basal oxygen consumption, without the addition of substrates or inhibitors; (ii) proton leak, after oligomycin (ATP-synthase blocker) addition, where oxygen consumption occurs due to mitochondrial inner membrane proton leak; (iii) uncoupled, after the addition of respiratory chain uncoupler carbonyl cyanide 3-chlorophenylhydrazone (CCCP), where the oxygen consumed reflects the maximal respiration rate (uncoupled to ATP synthesis); and (iv) inhibited, with the addition of rotenone (complex I blocker), and antimycin (complex III blocker), where the oxygen consumption reflects non-mitochondrial activity. The ECAR rate was verified through application of the glycolysis stress test kit (Agilent Technologies).

Another objective was to evaluate the influx of Na+into cells upon BREATHOX® POWDER treatment by fluorescence microscopy. Vero cells were stained using the intracellular sodium indicator Natrium Green and challenged with NaCl (3%) for the method calibration. Once the methodological conditions were established (45 min incubation in the presence of 11 μM Natrium Green), the cells were challenged with BREATHOX® POWDER (0.4, 0.8 and 1.1% wt/vol) and the Na+influx measured for 100 seconds. The changes in the Na+ concentrations upon BREATHOX® POWDER application were recorded by a Nikon fluorescence microscope coupled to a CCD camera and an automated shutter.

Based on the luciferase assay (scheme for measuring ATP concentration via luminescence production-FIG. 2), it is evident that at concentrations above 0.9% of BREATHOX® POWDER (i.e. 1.1, 1,2 and 1.4%) at 1 h and more evidently at 72 h cause a significant decrease in intracellular ATP concentration, being one mechanism to explain the decreased replication of SARS-COV-2 in Vero-cells.

In order to test whether the reduced level of ATP was caused by the increased activity of Na+/K+ ATPase transporter, Vero cells were treated with 0.4, 0.8 and 1.1% of BREATHOX® POWDER in presence or absence of 5 μM ouabain (Na+/K+ ATPase transporter inhibitor). Changes in total ATP concentration in Vero cells exposed to increasing concentrations of BREATHOX® POWDER with or without 5 μM ouabain for 1 hour was determined by plate microfluorimetry recordings with the FlexStation III microplate reader and the ATP Assay Kit (SIGMA-Aldrich) following the manufacturer's instructions. The SIGMA-Aldrich ATP Assay Kit provides sensitive results, detecting ATP release of 10-100 mammalian cells/well, based on the firefly luciferase-catalyzed oxidation of D-luciferin in the presence of ATP, in which the amount of ATP is quantified by the amount of light (hv) produced. Recordings of the luminescence intensity of 5×10⁴ cells in black clear-bottom 96-well plates were acquired at rest (point 0 h) and 1 h after BREATHOX® POWDER challenge (depolarizing agent). Time kinetics were obtained by measuring at 1.52 s intervals for 120 s after 10 s of monitoring basal fluorescence intensity, which is the luminescence emission rate prior to addition of the ATP-releasing agent. Responses were calculated as the peak luminescence minus the basal luminescence, using the SoftMax2Pro software (Molecular Devices). From the results obtained, FIG. 3 shows that the decrease in ATP level was significantly reversed with ouabain treatment.

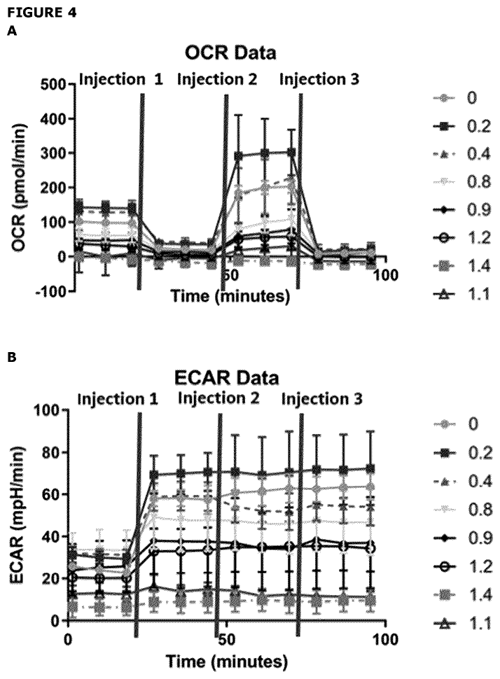

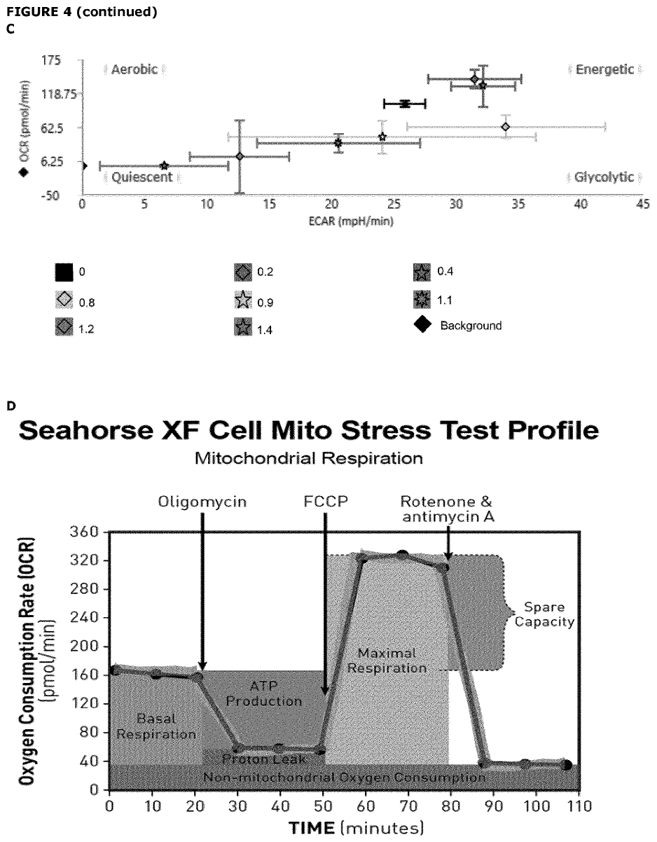

The mitochondrial status was also measured after every treatment condition, in order to assess the effect on ATP production. For this purpose, the Vero cells were tested by the Seahorse assay that measures the mitochondrial metabolism. Intact Vero cells were assessed with a high-resolution respirometry assay. For this, two parameters were evaluated: oxygen consumption rate (OCR; FIG. 4A) and extracellular acidification rate (ECAR; FIG. 4B). When mitochondrial metabolism is compromised, glycolytic enzymes enhance their activity to compensate for ATP production (FIG. 4D). The end product of this pathway is lactate, and its synthesis and accumulation results in medium acidification. FIG. 4D shows oxygen consumption in four states: (i) basal, (ii) proton leak, (iii) uncoupled, and (iv) inhibited. Basal indicates normal cell respiration values; proton leak reflects mitochondrial inner membrane integrity; uncoupled indicates respiratory chain activity of complex I to IV (i.e., maximal respiratory capacity); and inhibited reflects non-mitochondrial processes that consume oxygen. After 72 h of BREATHOX® POWDER addition in cultured Vero cells, up-regulated maximum respiration was found in 0.2% BREATHOX® POWDER in comparison to the control values. However, values above 0.8% BREATHOX® POWDER down-regulated respiration (FIG. 4A). In agreement, ECAR evaluation (FIG. 4B) indicated the same pattern. The correlation between OCR and ECAR is plotted in an energy map (FIG. 4C) that shows that 0.2% and 0.4% BREATHOX® POWDER treatment led to increased energetic state, while higher concentrations shift the metabolism to the anaerobic pathway. OCR and ECAR results indicate that lower concentrations of BREATHOX® POWDER are positive for the metabolism of the cells, while higher doses impair mitochondrial function in Vero cells. For any studies of chemicals with therapeutic potential, such a result is important since it is common to observe mitochondrial impairment during the development of new drugs.

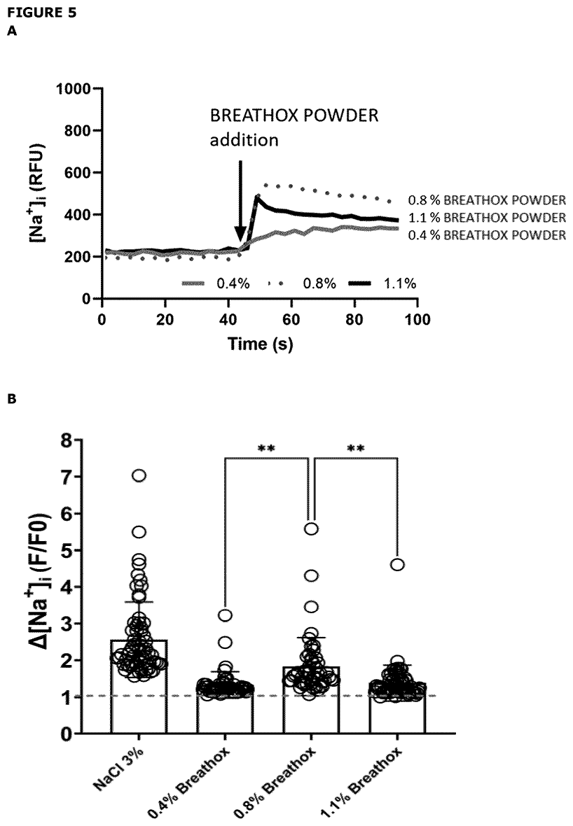

A further objective was to evaluate the influx of Na+upon BREATHOX® POWDER treatment by fluorescence microscopy. Vero cells were stained using the intracellular sodium indicator Natrium Green and challenged with NaCl (3%) for the method calibration. Once the methodological conditions were established (45 min incubation in the presence of 11 μM Natrium Green), the cells were after challenged with BREATHOX® POWDER (0.4, 0.8 and 1.1%) and the Na+influx measured for 100 seconds, as seen in FIGS. 5AB and 5B.

The Δ[Na+]i data shows that BREATHOX® POWDER application induces Na+inflow. At 0.8% BREATHOX® POWDER concentration, Na+inflow was significantly higher than at 0.4% and 1.1% BREATHOX® POWDER concentrations. This corroborates the data of the mitochondrial status, in which doses above 0.8% BREATHOX® POWDER were toxic for the mitochondria.

It is worth mentioning that 0% BREATHOX® POWDER corresponds to 0.6% NaCl concentration and 1.7% BREATHOX® POWDER corresponds to 2.1% NaCl, because BREATHOX® POWDER was diluted in cell culture medium containing 0.6% NaCl (110 mM NaCl).

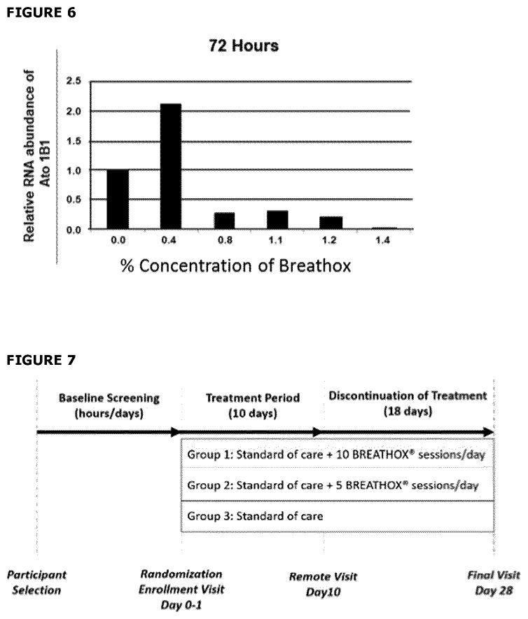

Gene expression of sodium channels in the cell culture was investigated by measuring the relative abundance of mRNA of Na+/K+ ATPase (Atp1B1, NCBI Reference Sequence: NM_001677.4); primers were standardised. As seen in FIG. 6, Na+/K+ ATPase expression was increased in 0.4% concentration of BREATHOX®, as a positive feedback to Na+/K+ ATPase overstimulation. However, doses higher than 0.8% BREATHOX® were toxic and decreased Na+/K+ ATPase channel expression. ENac expression in Vero cells was not observed.

Due to its ability to increase Na+/K+ ATPase transporter activity, BREATHOX® POWDER can induce a significant reduction in ATP level. Since SARS-COV-2 requires ATP for its replication, the experiments provided herein showed that BREATHOX® POWDER has significant properties in viral inhibition (replication inhibition) of SARS-COV-2.

### Example 3: Antiviral Effects Against COVID-19

An open-label, randomized, three-arm feasibility study was conducted, evaluating the effects of the BREATHOX® device comprising NaCl dry powder according to the present invention (specifically BREATHOX® POWDER as defined herein), in reducing COVID-19-related symptoms and preventing referral to health care services in adult patients diagnosed with COVID-19. Data were collected from 100 patients with COVID-19 who were randomized into three treatment groups.

The purpose of the study was to demonstrate the benefits of using BREATHOX® POWDER for reducing the severity of COVID-19 symptoms and preventing the deterioration and onset of further symptoms, based on the observed antiviral effects in the pre-clinical, in vitro study.

The study provides evidence of the feasibility of a full-scale trial of the effects of BREATHOX® POWDER in COVID-19 patients. The primary objective of the study was to determine the efficacy of using BREATHOX® comprising a NaCl dry powder composition of the present invention (BREATHOX® POWDER), as a dry powder inhaler therapy for clinical improvement in symptomatic, COVID-19 patients compared to standard of care (only treating/alleviating symptoms with paracetamol). Secondary objectives were to evaluate the efficacy of using BREATHOX® POWDER to reduce the use of health services and to assess the safety of adverse events after discontinuation of treatment within 28 days.

The outcomes were measured through remote medical monitoring of vital signs and symptoms, physical assessments, dairy data, and daily self-reporting of the participants. To evaluate the efficacy of BREATHOX® POWDER according to the objectives of the study and measure the development of the subjects' health status, an 8-point ordinal scale was used, as recommended by the World Health Organization (WHO) for measuring the health status of COVID-19 patients. The 100 participants who were included in the experiment were adult males or females (of 18 years of age or older), with a mild to moderate SARS-COV-2 infection, diagnosed by RT-PCR or swab antigen

PCR test. Their symptoms must be at least 1 of the following symptoms at inclusion: fever or fever perceived for more than 24 hours, headache, sore throat, cough (e.g. dry cough), fatigue, chest pain or choking sensation (no associated respiratory distress), myalgia, anosmia, ageusia, or gastrointestinal symptoms within 10 days of onset The patients included were not hospitalised. Subjects were randomised to a 1:1:1 ratio into three groups.

The two experimental treatment groups were Group 1 and Group 2. Group 1 received standard of care treatment combined with ten BREATHOX® sessions per day, meaning one session every hour during daytime, for ten days. Group 2 received standard of care combined with five BREATHOX® sessions per day, thus one session every three hours for ten days. Each participant in these two groups received two BREATHOX® devices, one for nasal and one for oral use. One BREATHOX® session consisted of 4 inhalations of the BREATHOX® POWDER using the BREATHOX® device, one in each nostril and two oral inhalations. Group 3 was the control group receiving only standard of care. The following is an overview of the three randomised groups:
Group 1: Standard of care+one BREATHOX® session every hour (4 inhalations/session), total of 10 sessions per day for 10 days-33 subjects.
Group 2: Standard of care+one BREATHOX® session every three hours (4 inhalations/session), total of 5 sessions per day 10 days-33 subjects.
Group 3: Only standard of care (no BREATHOX® sessions)—34 subjects.

The total duration of the study was 28 days with a treatment period of 10 days and the discontinuation of treatment for 18 days. At the first visit, informed consent forms were collected, vital signs were measured and a physical assessment was conducted by a health professional. After 10 days from study start, the patients received a remote visit by a telephone or video conversation to assess the use of the assigned treatment, adverse events and need for unscheduled medical care. During this call, the symptom diary was evaluated. After the visit, the treatment was discontinued for the remaining 18 days of the study. At the end of the study (Day 28), a final visit took place to evaluate presence of adverse events and collect symptoms diaries. This end-of-treatment visit took place virtually. FIG. 7 illustrates the study design.

The outcomes were measured based on RT-PCR COVID-19 tests, remote medical monitoring of vital signs and symptoms, diary data, and daily self-reporting of the participants. The principal investigator was responsible for ensuring the adherence to visits and follow-ups at the correct intervals. An Independent Safety Monitoring Committee was set up to continuously review and evaluate the clinical efficacy and safety data collected during the study at fortnightly intervals.

This feasibility study included 100 patients to have a first interim analysis of the efficacy and safety of the trial. All of the participants were analysed based on intention to treat analysis. The results of the study were analysed using analysis of variance (ANOVA) for continuous variables and the Kruskal-Wallis test for non-parametric variables. The proportion of the use of health resources and hospitalisation was calculated using the Chi-square test. The frequency of using health resources was assessed using Poisson Regression.

After analysing the data from the clinical trial, using Kaplan-Meier survival analysis, it was observed that the use of BREATHOX® POWDER during the treatment (both treatment groups) had a significant improvement compared with the control group in symptoms recovery. The duration of the cough induced by COVID-19, was significantly lower in the treatment groups.

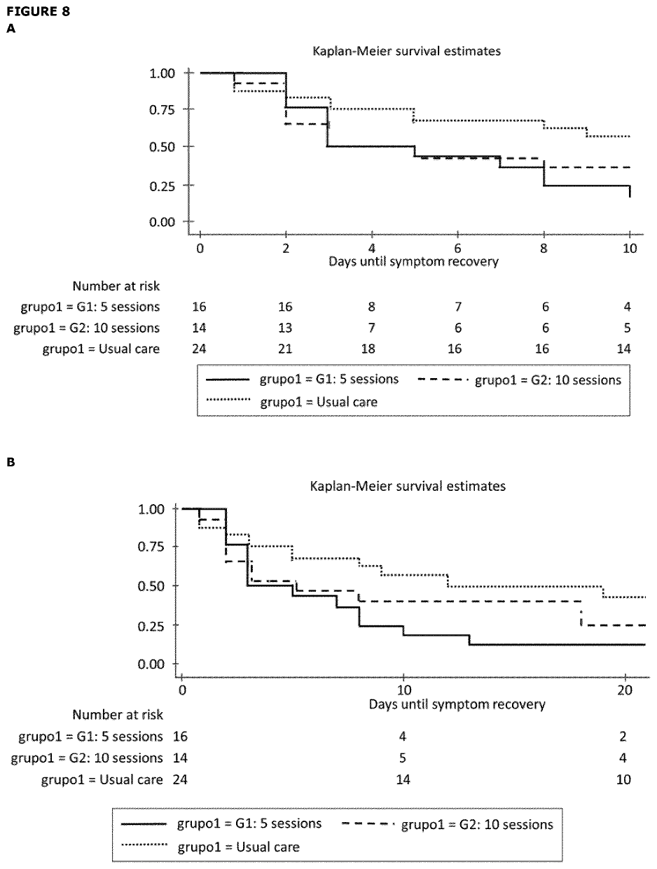

FIG. 8 shows the number of days it takes to recover from Cough as a symptom of Covid-19 affecting the airways. Only patients experience cough symptom at the beginning of the trial is included in this data set. The values reported below the graph in FIG. 8 represent of prevalence of symptoms—i.e. patients in each group experiencing cough symptoms; while in the graph, the y-axis shows the fraction of patients within each group experiencing cough (1.00=100%), at the time indicated on the x-axis.

As illustrated in FIG. 8, it was found that already 4 days after the first day of coughing:
50% of patients in group 1 and group 2 had recovered from Cough.
while only 25% of patients in group 3 had recovered from Cough.

Symptoms of cough was resolved 2 times faster in those who received BREATHOX® treatment compared to those submitted to usual care.

As further illustrated in FIG. 8, it was found that 10 days after the first day of coughing:
75% of patients in group 1 had recovered from Cough.
and 64% of patients in group 2 had recovered from Cough.
while only 42% of patients in group 3 had recovered from Cough.

As further illustrated in FIG. 8, it was found that 20 days after the first day of coughing:
88% of patients in group 1 had recovered from Cough.
and 72% of patients in group 2 had recovered from Cough.
while only 58% of patients in group 3 had recovered from Cough.

Hence, BREATHOX® comprising a NaCl dry powder of the present invention was demonstrated to reduce the recovery time of Cough in COVID-19 patients compared with standard of care.

Overall there were no adverse events reported related to the use of BREATHOX® POWDER and it was well tolerated by the patients.

Conclusion: The clinical study thereby confirmed what the in vitro findings showed, having found that the use of BREATHOX® comprising a NaCl dry powder of the present invention during treatment (both treatment groups) provided a significant (p=0.01) improvement compared to the control group in symptoms recovery. The resolution of COVID-19-induced cough was significantly shorter in the treatment groups. The ‘BREATHOX® group 1’ had 40% faster recovery of COVID-19-induced cough than the control group, and the ‘BREATHOX® group 2’ had 30% faster recovery than the control group (p=0.01).

The resolution of other COVID-19-induced symptoms was also seen to be improved, however not statistically significant due to low sample size.

1. A dry powder composition comprising at least 50 wt % NaCl for use in preventing and/or treating a viral respiratory tract infection, wherein said composition is administered to a mammalian subject by dry powder inhalation.

2. The dry powder composition for use according to claim 1, wherein said viral respiratory tract infection is caused by coronavirus.

3. The dry powder composition for use according to claim 1 or 2, wherein the composition reduces the time for resolution of one or more symptoms caused by the viral respiratory tract infection,

4. The dry powder composition for use according to any one of claims 1-3, wherein the one or more symptoms caused by the viral respiratory tract infection is cough.

5. The dry powder composition for use according to any one of claims 1-4, wherein the composition comprises at least 60% NaCl, preferably at least 70% NaCl, more preferably at least 80% NaCl, most preferably at least 90% NaCl.

6. The dry powder composition for use according to any one of claims 1-5, wherein the dry powder composition has a particle size distribution of at least 80% of the particles being in the range 1-10 μm.

7. The dry powder composition for use according to any one of claims 1-6, wherein the composition further comprises an anti-agglomerating agent.

8. The dry powder composition for use according to any one of claims 1-7, wherein the sole therapeutic agent of the composition is said NaCl.

9. The dry powder composition for use according to claim 7, wherein the sole therapeutic agents are said NaCl and said anti-agglomerating agent.

10. The dry powder composition for use according to any one of claims 7-9, wherein the anti-agglomerating agent is a carbohydrate or carbohydrate derivative.

11. The dry powder compositions for use according to any one of claims 7-10, wherein the anti-agglomerating agent is selected from lactose, mannitol, and maltodextrin; preferably lactose.

12. The dry powder composition for use according to any one of claims 1-11 wherein the composition is administered in a daily dosage of between 0.5-200 mg.

13. The dry powder composition for use according to claim 12, wherein the daily dosage is divided into 1-10 daily sessions of 4-10 mg per session.

14. The dry powder composition for use according to any one of claims 1-13, wherein the composition is administered orally and/or nasally.

15. The dry powder composition for use according to any one of claims 1-14, wherein the composition substantially consists of NaCl and lactose.

16. The dry powder composition for use according to any one of claims 1-15, wherein the composition comprises lactose, and wherein the NaCl to lactose ratio is between 99:
1 and 75:25 based on wt %.

17. The dry powder composition for use according to claim 16, wherein the NaCl to lactose ratio is approximately 95:5 based on wt %.

18. The dry powder composition for use according to any one of claims 2-17, wherein the coronavirus is SARS-COV-2.

19. The dry powder composition for use according to any one of claims 2-18, wherein the viral respiratory tract infection is COVID-19.

20. The dry powder composition for use according to any one of claims 1-19, wherein the mammalian subject is a human.

21. The dry powder composition for use according to any one of claims 4-20, wherein the composition reduces the time for resolution of cough of the mammalian subject by at least 30% compared to a mammalian subject not being treated with the dry powder composition.

22. The dry powder composition for use according to any one of claims 1-21, wherein the NaCl is provided by a composition obtained from the Hvornum Salt Diapir (N56 36.834 E009 42.070) in Denmark.

23. The dry powder composition for use according to any one of claims 1-22, wherein the composition is administered using a dry powder inhaler.

24. The dry powder composition for use according to claim 23, wherein the dry powder inhaler is an inhaler (1) having in an axial direction a proximal end (P) for insertion into the mouth of a user, and a distal 40 end (D) opposite to the proximal end (P), wherein the inhaler (1) comprises an inlet (4), an outlet (5) arranged at the proximal end (P), an air passage (10, 11, 12, 13) extending from the inlet (4) to the outlet (5), and a reservoir (8, 9) communicating with the air passage (10, 11, 12, 13) through a release orifice (6, 7), the reservoir (8, 9) containing a dispersible substance, wherein the inhaler (1) has a proximal part (2) comprising the outlet (5), and a distal part (3) attached to the proximal part (2), wherein the proximal part (2) is linearly slidable along the axial direction (A) with respect to the distal part (3) between an ‘OPEN’ position where the proximal part (2) is deployed from the distal part (3), and a ‘CLOSED’ position where the proximal part (2) is retracted towards the distal part (3), wherein the inhaler (1) further comprises an inlet valve member (14), and an outlet valve member (15);
wherein the reservoir (8, 9) is arranged in the proximal part (2);
wherein the inlet (4) comprises one or more openings in a peripheral housing wall (20) of the proximal part (2), the one or more openings facing radially outward, away from the axial direction (A), and the inlet valve member (14) is formed by a peripheral housing wall (30) of the distal part (3) covering the openings when the proximal part (2) is in the ‘CLOSED’ position;
wherein the outlet (5) comprises an axially oriented aperture,
wherein the outlet valve member (15) is formed as a plug attached to the distal part (3), the plug blocking the outlet (5) when the proximal part (2) is in the ‘CLOSED’ position;
wherein the release orifice (6, 7) is oriented in the axial direction (A); and
wherein the inhaler (1) further comprises a reservoir valve member (16, 17), wherein the reservoir valve member (16, 17) is formed as a peg travelling in the axial direction (A), the peg being fixed to the distal part (3) via an axially extending stem, wherein the peg blocks the release orifice (6, 7) when the proximal part (2) is in the ‘CLOSED’ position;
whereby said inlet valve member (14), outlet valve member (15) and reservoir valve member (16, 17) are arranged so as
to simultaneously close the inlet (4), the outlet (5), and the release orifice (6, 7) when the proximal part (2) is retracted towards the distal part along the axial direction from the ‘OPEN’ position into the ‘CLOSED’ position, and
to simultaneously open the inlet (4), open the outlet (5), and deliver an amount of the dispersible substance from the reservoir (8, 9) through the release orifice (6, 7) to the air passage (10, 11, 12, 13) when the proximal part (2) is deployed from the distal part along the axial direction from the ‘CLOSED’ position into the ‘OPEN’ position.
